(19) United States
(12) Patent Application Publication
(10) Pub. No.: US 2021/0085187 A1
(43) Pub. Date: Mar. 25, 2021

(54) ORAL SCREENING DEVICE HAVING A REPLACEABLE SCREENING HEAD

(71) Applicant: ASIA VITAL COMPONENTS Co., LTD., New Taipei City (TW)

(72) Inventors: Wei-Chuan Wang, New Taipei City (TW); Ting-Yu Lin, New Taipei City (TW); Chung-Liang Chiang, New Taipei City (TW)

(21) Appl. No.: 16/576,791

(22) Filed: Sep. 20, 2019

(51) Int. Cl. A61B 5/00; A61B 1/06; A61B 1/05

(52) U.S. Cl. CPC A61B 5/0088; A61B 1/043; A61B 1/053; A61B 1/0646

(57) ABSTRACT

An oral screening device having a replaceable screening head is provided that comprises a screening head provided with a camera module and a handle from which the screening head is detachable. The camera module is disposed in the screening head. The handle is provided with a control unit disposed in the handle. The control unit is provided with a controller electrically coupled with the camera module. Through this design of the present invention, easy replacement of the screening head is achieved.

### BACKGROUND OF THE INVENTION

### 1. Field of the Invention

The present invention relates to an oral screening device, and in particular to an oral screening device having a replaceable screening head that enables easy replacement of the screening head.

### 2. Description of the Related Art

Oral health has a significant impact on people's lives, and the oral cavity is the only area in the human body that exposes organs such as the teeth, gums, and tongue. So, periodic oral examination is a very important thing. As medicine has developed over time, oral examination for the organs inside the mouth is not only for examining the condition of the teeth but for predicting that whether a lesion occurs in the body, which facilitates more and more applications. For the teeth in the oral cavity, the dental plaque accumulating on the mouth is a community made of different species of bacteria that is barely visible by the human eye, and, without a medical grade oral screening device, can only be diagnosed by a professional dentist. The dentist, however, mainly uses his/her eyes to examine the oral cavity, and the diagnosis made by the dentist may vary depending on different factors such as the experiences of the dentist. The dentist is also unable to specifically point out the distribution of dental plaque, caries, and calculus. These reasons result in inaction for timely treatment and prevention. The caries has always reached its late-stages when a patient can feel a tooth pain and the pain can be easily examined by visual and tactile detection, and thus causes damage to the teeth if not treated in time. Although the dental plaque and calculus on the teeth can be detected by a large dental equipment with medical grade teeth screening devices, the cost can be very expensive, resulting in a certain financial pressure. Therefore, there is a need for a device that enables a user to self-check his/her mouth in order to have a basic understanding about the condition of his/her own oral cavity.

### SUMMARY OF THE INVENTION

An object of the present invention is to provide an oral screening device having a replaceable screening head that enables easy replacement of the screening head.

Another object of the present invention is to provide an oral screening device having a replaceable screening head that includes a screening head detachable from a handle, so that diversity in oral screening can be achieved by replacing screening heads with different functions.

Another object of the present invention is to provide an oral screening device having a replaceable screening head that achieves cost reduction, convenience of use, and medical hygiene.

To achieve the above-mentioned objects, the present invention is provided with an oral screening device having a replaceable screening head comprising a screening head and a handle. The screening head is provided with a camera module that is disposed in the screening head, and the screening head is detachable from the handle. The handle is provided with a control unit disposed in the handle, and the control unit is provided with a controller electrically coupled with the camera module. Through the design of the oral screening device having a replaceable screening head of the present invention, easy replacement of the screening head, diversity in oral screening, and cost reduction can be achieved.

### BRIEF DESCRIPTION OF THE DRAWINGS

FIG. 1A is an exploded perspective view of an embodiment of the invention;

FIG. 1B is an exploded perspective view of the embodiment of the invention in another angle;

FIG. 2 is a perspective view of the embodiment of the invention;

FIG. 3 is a block diagram of the embodiment of the invention;

FIG. 4 is an illustration showing that a user operates an oral screening device connected with an external interface according to the embodiment of the invention;

FIG. 5 is an illustration showing that an oral screening device of the embodiment of the invention is connected to an external interface;

FIG. 6 is a block diagram of an alternative embodiment of the invention; and

FIG. 7 is a block diagram of another alternative embodiment of the invention.

### DETAILED DESCRIPTION OF THE PREFERRED EMBODIMENTS

The above-mentioned objects, and the structure and functions of the present invention are to be illustrated with reference to the preferred embodiments in the accompanying drawings.

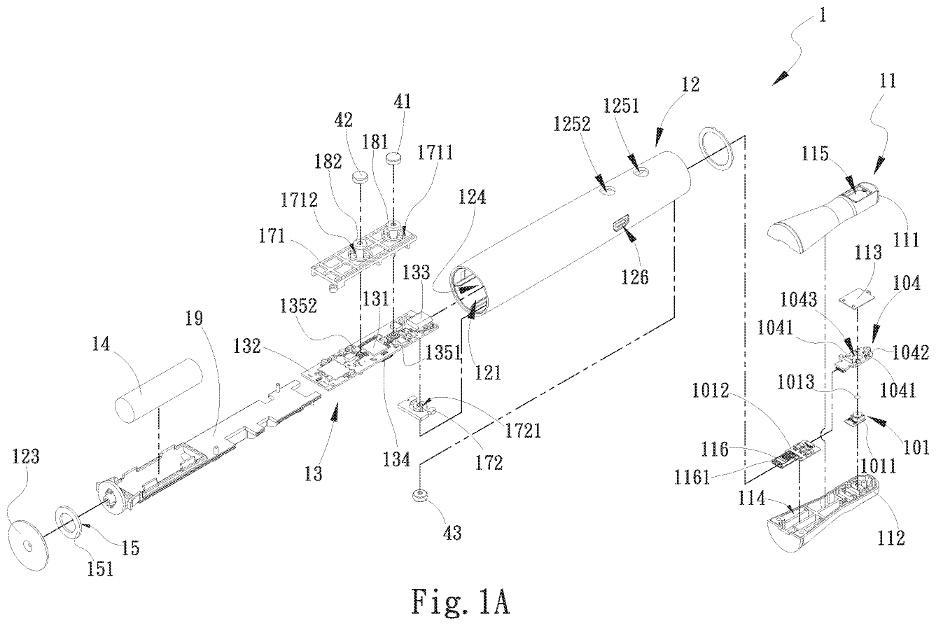

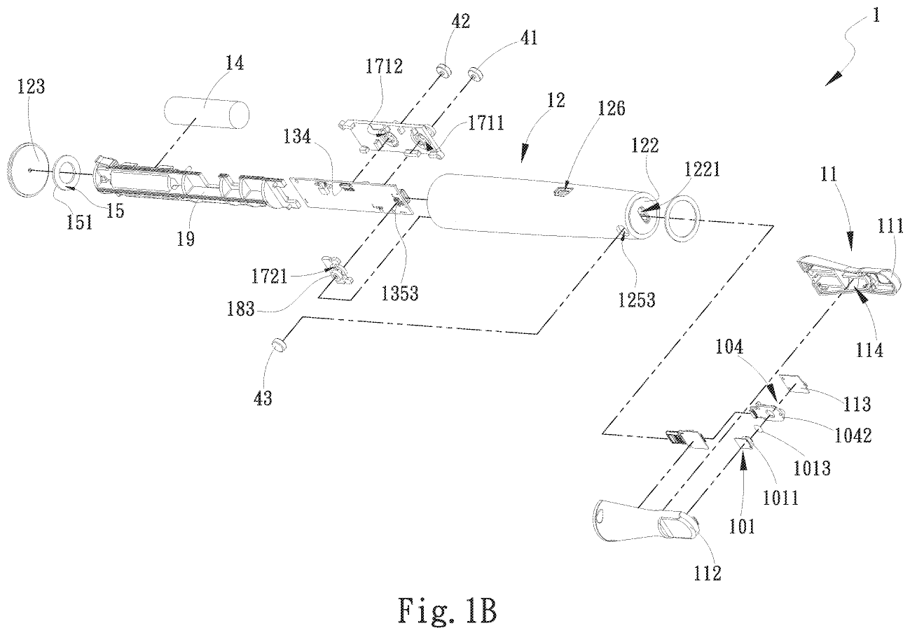

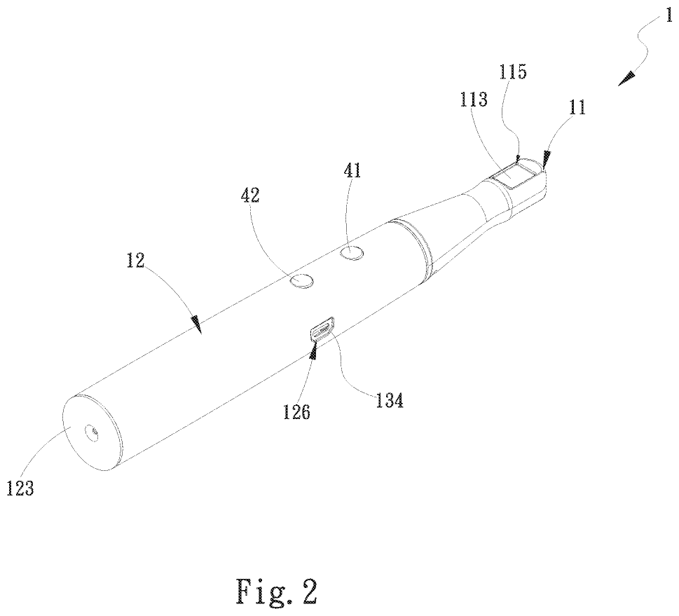

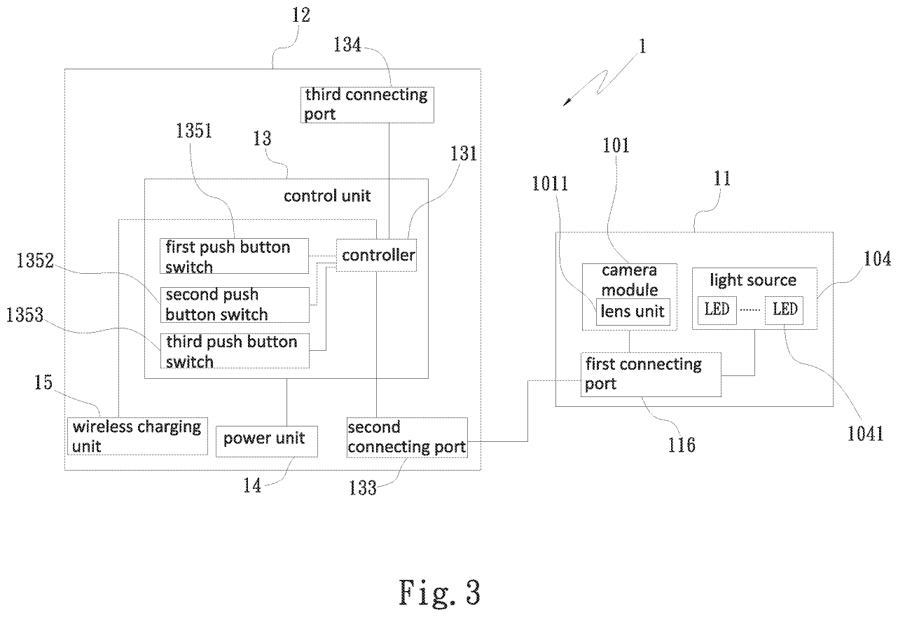

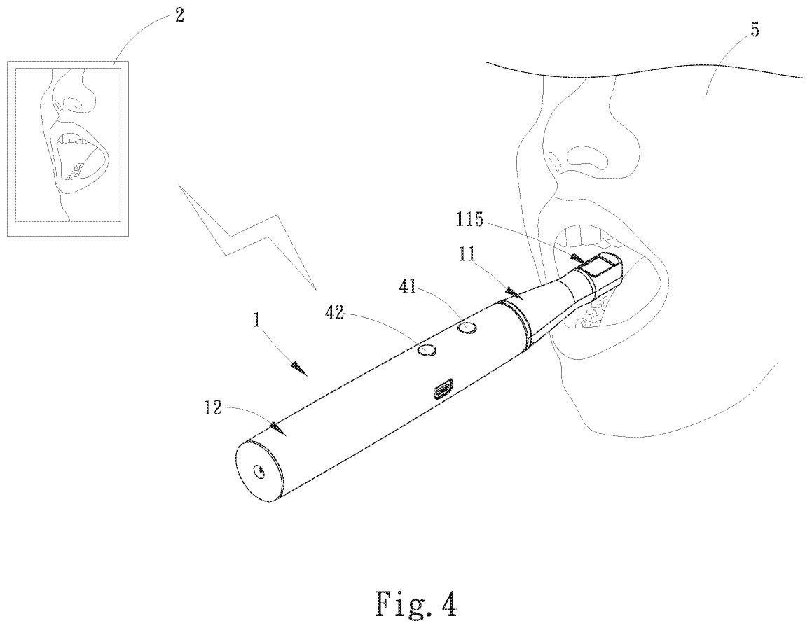

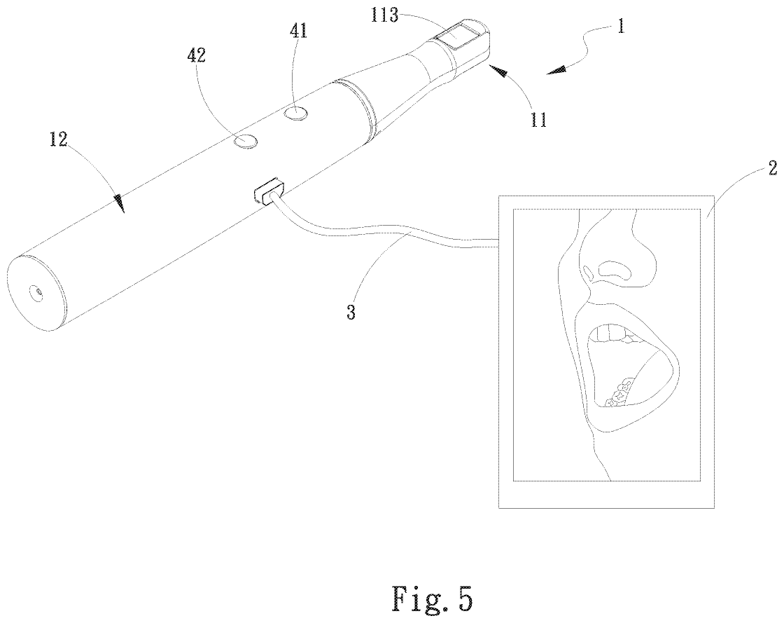

The present invention is provided with an oral screening device having a replaceable screening head with reference to FIG. 1A being an exploded perspective view of an embodiment of the invention, FIG. 1B being an exploded perspective view of the embodiment of the invention in another angle, FIG. 2 being a perspective view of the embodiment of the invention, FIG. 3 being a block diagram of the embodiment of the invention, FIG. 4 being an illustration showing that a user operates an oral screening device connected with an external interface according to the embodiment of the invention, and FIG. 5 being an illustration showing that an oral screening device of the embodiment of the invention is connected to an external interface. As shown in the FIGS., the oral screening device 1 is suitable for detection of the oral cavity of a user 5. The oral screening device 1 includes a screening head 11 and a handle 12. The screening head 11 is detachable from the handle 12, and in this embodiment, is detachable from the handle 12 in an insertion manner, forming the screening device 1 having the replaceable screening head 11, which is not limited to this embodiment. In other embodiment, manners capable of detaching the screening head 11 from the handle 12, and attaching the detached the screening head 11 back to the handle 12 can be the detachable connection relationship between the screening head 11 and the handle 12 described in the present invention. For example, the screening head 11 is detachable from the handle 12 in the manner of a screw.

The screening head 11 is provided with an opening 115, a first connecting port 116, an upper cover 111, a lower cover 112, a camera module 101, a set of light sources 104, and a transparent protective plate 113. The opening 115 is disposed on one end of the screening head 11, while the first connecting port 116 is disposed on the other end of the screening head 116. In this embodiment, the opening 115 is disposed on a top surface of the upper cover 111, and the first connecting port 116 is disposed on a bottom surface of the screening head 11 and is electrically coupled to the camera module 101 and the set of light sources 104. Furthermore, the opening 115 is covered with the transparent protective plate 113 used to protect the camera module 101 and the set of light sources 104 inside the opening 115. The transparent protective plate 113 is also used for waterproofing, dust proofing and anti-fogging. The upper and lower covers 111 and 112 are combined together to jointly define a cover space 114.

The camera module 101 and the set of light sources 104 are disposed in the screening head, and in this embodiment, are disposed in the cover space 114 corresponding to the opening 115. The camera module 101 is provided with a camera circuit board 1012, a lens unit 1011, and a filter 1013. The camera circuit board 1012 is a printed circuit board (such as a flexible printed circuit board or rigid printed circuit board). The lens unit 1011 with 250-1000 nm spectral response in the cover space 114 is disposed on an end of camera circuit board 1012 that aligns with the opening 115, and is electrically coupled with the camera circuit board 1012. And, the lens unit 1011 is used to take an oral image (the condition of the teeth, throat, or tongue within the oral cavity) of a user 5 via the opening 115 when he/she is opening his/her mouth. The first connecting port 116 is disposed on one side of the camera circuit board 1012 and electrically coupled therewith, and an insertion end 1161 of the first connecting port 116 extends from the camera circuit board 1012 and away from the bottom surface of the screening head 11. In other embodiment, the insertion end 1161 of the first connecting port 116 extends from the camera circuit board 1012 and disposed within the bottom surface of the screening head 11.

The filter 1013 is a filter that transmits light having wavelengths greater than or equal to 490 nm and less than or equal to 650 nm to filter out light having a wavelength outside a certain period of wavelengths (e.q., light outside the wavelengths from 405-470 nm). In this embodiment, the filter 1013 is disposed in front of a lens of the lens unit 1011. In other embodiment, the filter 1013 may be disposed between an image sensor and the lens, both of which are in the lens unit 1011.

The set of light sources 104 is disposed between the camera module 101 and the opening 115, and is provided with a plurality of light emitting diodes (LEDs) 1041 and a light source circuit board 1042. The light source circuit board 1042 is provided with a hole 1043 through which the perimeter of the lens unit 1011 of the camera module 101 passes, and the plurality of light emitting diodes 1041 is thus located around the camera module 1011. The plurality of light emitting diodes 1041 can be blue light emitting diodes, white light emitting diodes, violet light emitting diodes, or any combination thereof. The plurality of light emitting diodes 1041 is disposed on a side of the light source circuit board 1042, and electrically coupled to each other.

In this embodiment, the plurality of light emitting diodes 1041 is embodied as a plurality of blue light emitting diodes 1041 and a plurality of white light emitting diodes 1041, which are disposed adjacent to the hole 1043 on the side of the light source circuit board 1042, and aligns with the opening 115. Accordingly, the light emitted by the plurality of blue light emitting diodes 1041 and the plurality of white light emitting diodes 1041 passes through the opening 115, wherein the plurality of blue light emitting diodes 1041 having a wavelength ranging from 360 nm to 470 nm. In this embodiment, the blue light emitting diodes 1041 controlled by the light source circuit board 1042 have two different wavelengths, i.e., one is 405 nm, which is for screening dental plaque, and the other is 470 nm, which is for screening calculus or caries. Therefore, when the plurality of blue light emitting diodes 1041 is used as a screening light, the lights (i.e., blue lights) emitted by the blue light emitting diodes 1041 illuminates the mouth of a user, and is reflected to the lens unit 1011 of the camera module 101 passing through the filter 1013. The lens unit 1011 only captures the fluorescence (i.e., red fluorescence) reflected by a mass of bacteria (such as dental plaque, calculus, or caries) that grows on surfaces of the teeth within the mouth, and forms an image. So, the user 5 can know whether the dental plaque, calculus, caries, infection, or inflammation is on or around the teeth, and a diagnosis is made accordingly. Because the filter 1013 filters the blue light reflected to the lens unit 1011, the red fluorescence is the only fluorescence shown in the image of the lens unit 1011, thus making the image clearer and more visible.

When the plurality of white light emitting diodes 1041 is used as a screening light and the plurality of blue light emitting diodes 1041 does not emit light, the filter 1013 does not overlap the lens unit 1011, which allows the lights emitted by the white light emitting diodes 1041 to illuminate the mouth, and to be reflected to the lens unit 1011 of the camera module 101 directly. The lens unit 1011 takes an image of the mouth, so that the user 5 can be clearly aware of the condition (such as tonsillitis or glossitis) of his/her own mouth.

Referring to FIGS. 1A, 1B, and 5, the handle 12 is provided with an open end 121, closed end 122, sealing component 123, first button hole 1251, second button hole 1252, third button hole 1253, power unit 14, and control unit 13. The power unit 14 in this embodiment is a rechargeable battery, and is electrically coupled to the control unit and camera module 101 for power supplying. The open end 121 and closed end 122 define a handle space 124 used for the placement of a holder bracket 19. The sealing component is attached to the open end 121 and provides a seal therebetween to effectively achieve waterproof and dust proof effects. The closed end 122 is provided with a connecting hole 1221 in communication with the handle space 124 that is used for insertion of the first connecting port 116 of the screening head 11. The first, second, and third button holes 1251, 1252, and 1253 are formed on the handle 12. In this embodiment, the first and second button holes 1251 and 1252 are disposed on one side of the handle 12, while the third button hole 1253 is disposed on the other side of the handle 12. The control unit 13 disposed in the handle 12 is provided with a controller 131, control circuit board 132, second connecting port 133, first push button switch 1351, second push button switch 1352, and third push button switch 1353. The control circuit board 132 is a printed circuit board (such as a flexible printed circuit board or rigid printed circuit board). In this embodiment, the control circuit board 132 and power unit 14 are placed on a side of the holder bracket 19, the power unit 14 is electrically coupled with the circuit board 132, the control circuit board 132 and power unit 14 on the holder bracket 19 are disposed in the handle space 124 of the handle 12, and the second connecting port 133 is disposed on one side of the control circuit board 132 that aligns with the connecting hole 1221, and electrically coupled with the controller 131. Accordingly, the first connecting port 116 of the screening head 11 can be connected to the corresponding second connecting port 133 via the connecting hole 1221, or the first connecting port 116 can be detached from the second connecting port 133 and away the connecting hole 1221. In implementation, the controller unit 13 is provide with a memory (not shown) electrically coupled with the controller 131 that is used to store images (such as oral images) taken. The memory can be a cache and random access memory (RAM).

In addition, the handle 12 is provide with a third connecting port 134 that is disposed on the other side of the control circuit board 132 and electrically coupled therewith. The third connecting port 134 aligns with an insertion hole 126 on an another side of the handle 12, and the third connecting port 134 is used for connecting a separable external interface 2. The external interface 2 can be a monitor (such as LCD and OLED), electronic device having a display (such as a phone, tablet, and computer), or power supply. When the oral screening device 1 is used for oral screening, the controller 131 controls the taken oral images to be transmitted to the external interface 2 (a monitor) through wired communication (a signal cable 3 for data transmission or data and power transmission) via the third connecting port 134, and to be displayed on the monitor, as shown in FIG. 5. The user 5 can therefore see the images of his/her own mouth. When the oral screening device 1 needs to be recharged, the controller 131 controls the power to be transmitted from the external interface 2 to the power unit 14 through the signal cable 3 via the third connecting port 134. In this embodiment, the first, second, and third connecting ports 114, 133, and 134 are embodied, but not limited as, a USB port. In implementation, the first, second, and third connecting ports 114, 133, and 134 can be mini USB ports, micro USB ports, or USB Type-C ports.

The controller 131 is, but not limited to, a microcontroller unit (MCU) 131. In implementation, the controller 131 can be a central processing unit (CPU), system on a chip, digital signal processor (DSP), or other controller 131 for image signal and data processing. The controller 131 is electrically coupled to the camera module 101, and is disposed on one side of the control circuit board 132 and electrically coupled therewith. The controller 131 receives the oral images (such as images of dental plaque, tonsillitis, or glossitis) taken by the lens unit 1011, which is transmitted by the first connecting 116, via the second connecting port 133, and transmits, through the signal cable 3, the oral images to the external interface 2 for displaying. The first and second push button switches 1351 and 1352 are disposed on one side of the control circuit board 132 that align with the first and second button holes 1251 and 1252, respectively, and are electrically coupled with the control circuit board 132 and controller 131. In this embodiment, the first and second push button switches 1351 and 1352 are embodied as a power switch and mode switching switch, respectively, wherein the power switch (i.e., the first push button switch 1351) is used to turn the oral screening device 1 on and off, while the mode switching switch (i.e., the second push button switch 1352) is used to switch between modes of the oral screening device 1, e.q., a dental plaque screening mode by pressing the mode switching switch once, a calculus and caries screening mode by pressing the mode switching switch twice, and an oral illumination mode by pressing the mode switching switch three times.

The third push button switch 1353 is disposed on the other side of the control circuit board 132 that aligns with the third button hole 1253, and electrically coupled therewith. In this embodiment, the third push button switch 1353 is embodied as a filming switch that starts to film a video. A first button bracket 171 is disposed on one side of the control circuit board 132 that has a first through hole 1711 and second through hole 1712. The first through hole 1711 allows a first pillar 181 to move up and down therethrough, while the second through hole 1712 allows the second pillar 182 to move up and down therethrough. The top surfaces of the first pillar 181 and second pillar 182 are covered with a first switch cap 41 and second switch cap 42, respectively. The upper portions of the first pillar 181 and second pillar 182, and the first and second switch caps 41 and 42 thereon pass through the first and second button holes 1251 and 1252, respectively. The bottom surfaces of the first pillar 181 and second pillar 182 are in contact with the top surfaces of the first and second push button switches 1351 and 1352, respectively.

Therefore, when the first switch cap 41 is pressed, the first pillar 181 (or the second pillar 182) drives the corresponding first push button switch 1351 (or the second push button switch 1352) to be turned on so as to turn the oral screening device on, and when the first switch cap is pressed again, the first pillar 181 drives the corresponding first push button switch 1351 to be turned off so as to turn the oral screening device off. when the second switch cap 42 is pressed after the oral screening device 1 is turned on, the second pillar 182 drives the corresponding second push button switch 1352 to be turned on so that the controller 131 controls the oral screening device to perform different screening modes (dental plaque screening mode, calculus and caries screening mode, and oral illumination mode). It is noted that if the oral screening device 1 is not running (turned off), the second and a third switch caps 42 and 43 do not drive the corresponding push button switches to be turned on when pressed.

In addition, a second button bracket 172 is disposed on the other end of the control circuit board 132 that has a third through hole 1721. The third through hole 1721 allows the third pillar 183 to move up and down therethrough, and the top surface of the third pillar 183 is covered with the third switch cap 43. The upper portion of the third pillar 183 and the third switch cap 43 thereon pass through the third button hole 1253. The bottom surface of the third pillar 183 is in contact with the top surface of the third button switch 1353. Therefore, when the third switch cap 43 is pressed after the oral screening device 1 is turned on, the third pillar 183 drives the corresponding third push button switch 1353 to be turned on so that the controller 131 controls the lens unit to take an image (i.e., oral image).

The handle 12 further comprises a wireless charging unit 15 that is provide with a wireless charging coil 151 electrically coupled with the control circuit board 132 and controller 131. The wireless charging coil 151 is disposed between the sealing component 123 and control circuit board 132 in the handle 12, which is used to be inductive coupled with a corresponding wireless charging coil in the wireless charging station (not shown). The inductive coupling generates an electromagnetic field after the coil in the wireless charging station is turned on, and allows the wireless charging coil 151 in the handle 12 to take power from the electromagnetic field and convert it back into electric current. The current is then converted into direct current, and transmitted to the power unit 14 and saved.

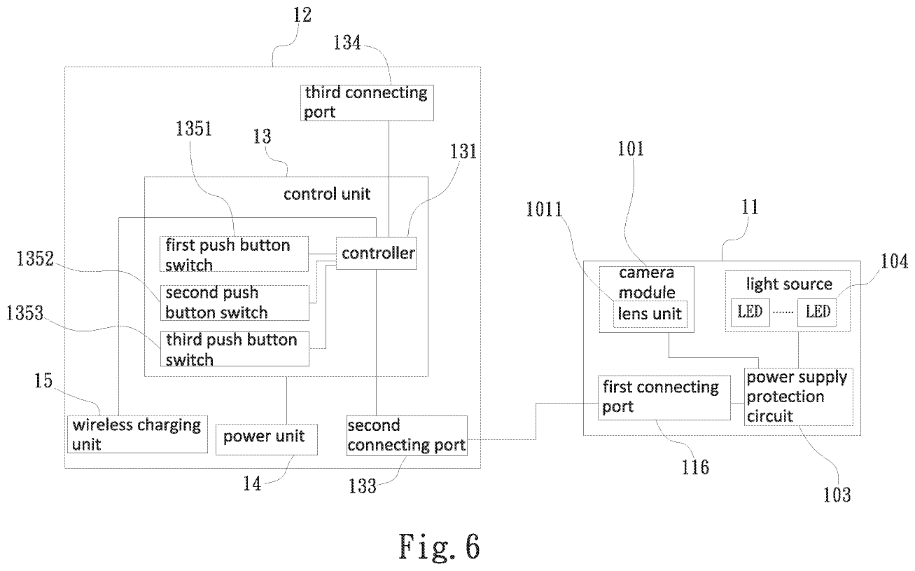

In an alternative embodiment, referring to FIG. 6 (using FIG. 1A as an auxiliary), the screening head 11 is provided with a power supply protection circuit 103 disposed on one side of the camera circuit board 1012. The power supply protection circuit 103 is electrically coupled with the camera module 101 and the set of light sources 104 as well as the first connecting port 116. The power supply protection circuit 103 permits the power to be transmitted to the camera module 101 and the set of light sources 104 when the first connecting port 116 is in electrical communication with the second connecting port 133, so the controller 131 can controls the camera module 101 and the set of light sources 104 to perform their corresponding screening modes. On the other hand, the power supply protection circuit 103 do not permit the power to be transmitted to the camera module 101 and the set of light sources 104 when the first connecting port 116 is not in electrical communication with the second connecting port 133, so the controller 131 cannot control the camera module 101 and the set of light sources 104 to perform their corresponding screening modes. Accordingly, a safety mechanism can be achieved. Where the first connecting port 116 is in electrical communication with the second connecting port 133 means the first connecting port 116 is connected to and is electrically coupled with the second connecting port 133, and where the first connecting port 116 is not in electrical communication with the second connecting port 133 means the first connecting port 116 is not well connected to and not electrically coupled with the second connecting port 133, or the first connecting port 116 is not connected to and not electrically coupled with the second connecting port 133.

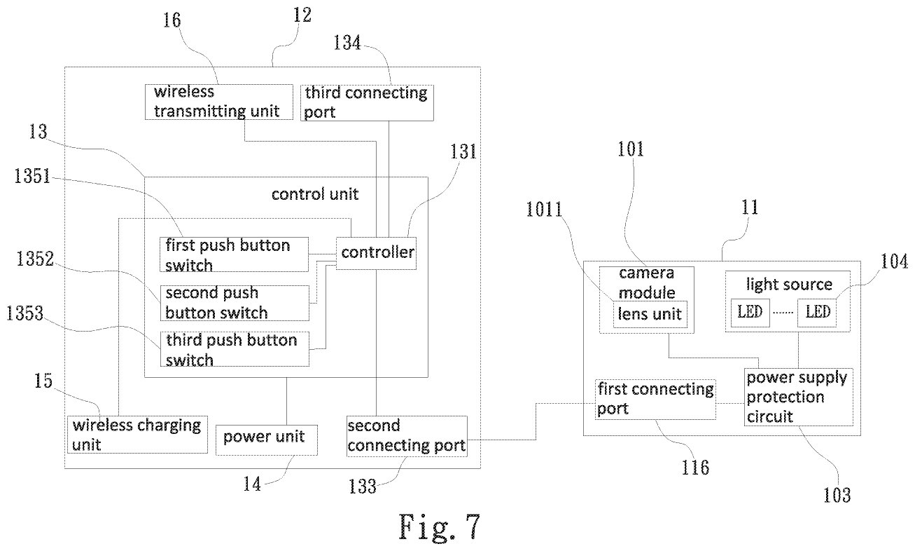

In another alternative embodiment, referring to FIG. 7 (using FIGS. 1A and 4 as auxiliaries), the handle 12 further comprises a wireless transmitting unit 16. The wireless transmitting unit 16 such as a wireless transmitter is disposed on one side of the control circuit board 132 and used to transmit the oral images. The wireless transmitting unit 16 is electrically coupled with the control circuit board 132 and controller 131. The controller 131 sends the received oral images to the external interface 2 such as an electronic device via wireless communication (such as Bluetooth, Wi-Fi, Near-Field Communication (NFC), 3G, 4G, and 5G), and the oral images thus are displayed on the electronic device (FIG. 4).

Therefore, by the detachable design of the screening head 11 with the handles 12, the user 5 can replace different screening heads 11 with different functions on the handle 12 depending on different purposes to conduct oral screening, achieving diversity in the oral screening. Furthermore, replacing the used screening head 11 on the handle 12 by a new screening head 11, the user can then perform oral screening for another person without purchasing a new set of the screening device. The present invention achieves the easy replacement of the screening head, convenience of use, and cost reduction, and further achieves medical hygiene.

What is claimed is:

1. An oral screening device having a replaceable screening head, comprising:
a screening head provided with a camera module that is disposed in the screening head; and
a handle from which the screening head is detachable, the handle to provided with a control unit disposed in the handle, the control unit provided with a controller electrically coupled with the camera module.

2. The oral screening device having a replaceable screening head according to claim 1, wherein the screening head is provided with a set of light sources disposed in the screening head, and the set of light sources is provided with a plurality of light emitting diodes that is located around the camera module.

3. The oral screening device having a replaceable screening head according to claim 2, wherein the screening head is provided with an upper cover, a lower cover, an opening, a first connecting port, and a transparent protective plate, the opening is disposed on a top surface of the upper cover and is covered with the transparent protective plate, the upper cover and the lower cover are combined together to jointly define a cover space in which the set of light sources and the camera module are disposed, and the first connecting port is electrically coupled with the camera module and the set of light sources.

4. The oral screening device having a replaceable screening head according to claim 3, wherein the camera module is provided with a camera circuit board and a lens unit, the lens unit in the cover space is disposed on one side of the camera circuit board that aligns with the opening and is electrically coupled therewith, the lens unit takes an oral image of a user via the opening and sends the oral image to the controller, the first connecting port is disposed on one side of the camera circuit board, and an insertion end of the first connecting port extends from the camera circuit board and away from a bottom surface of the screening head or the insertion end extends from the camera circuit board and is disposed within the bottom surface of the screening head.

5. The oral screening device having a replaceable screening head according to claim 4, wherein the camera module is provided with a filter that filters out light having a wavelength outside a certain period of wavelengths, the filter is disposed in front of or behind a lens of the lens unit, and the plurality of light emitting diodes is a plurality of blue light emitting diodes, the lights of which is reflected to the lens unit passing through the filter.

6. The oral screening device having a replaceable screening head according to claim 1, wherein the handle is provided with a power unit that is disposed in the handle, and is electrically coupled with the control unit and the camera module for power supplying.

7. The oral screening device having a replaceable screening head according to claim 3, wherein the handle is provided with a first button hole, a second button hole, a third button hole, and a second connecting port, the first, second, and third button holes are disposed on the handle, the control unit is provided with a first push button switch, a second push button switch, a third push button switch, and a control circuit board, the first and second push button switches are disposed on one side of the control circuit board that align with the first and second button holes, respectively, and are electrically coupled with the control circuit board, the third push button switch is disposed on the other side of the control circuit board that align with the third button hole and is electrically coupled with the control circuit board, and the second connecting port is disposed on one side of the control circuit board and is electrically coupled with the controller for connecting with the corresponding first connecting port.

8. The oral screening device having a replaceable screening head according to claim 7, wherein a first button bracket is disposed on one side of the control circuit board that has a first through hole and second through hole, the first through hole allows a first pillar to move up and down therethrough, the second through hole allows the second pillar to move up and down therethrough, upper portions of the first pillar and the second pillar pass through the first and second button holes, respectively, and bottom surfaces of the first pillar and the second pillar are in contact with top surfaces of the first and second push button switches, respectively.

9. The oral screening device having a replaceable screening head according to claim 8, wherein a second button bracket is disposed on the other side of the control circuit board that has a third through hole, the third through hole allows a third pillar to move up and down therethrough, an upper portion of the third pillar passes through the third button hole, and a bottom surface of the third pillar is in contact with a top surface of the third push button switch.

10. The oral screening device having a replaceable screening head according to claim 1, wherein the handle is provided with a wireless charging unit that is provided with a wireless charging coil, the wireless charging coil is disposed in the handle and is electrically coupled with the controller for inductive coupling with a corresponding wireless charging coil of a wireless charging station.

11. The oral screening device having a replaceable screening head according to claim 7, wherein the screening head is provided with a power supply protection circuit that is disposed on one side of the camera circuit board and is electrically coupled therewith, and the power supply protection circuit allows the camera module and the set of light sources to be enabled when the first connecting port is in electrical communication with the second connecting port, and does not allow the camera module and the set of light sources to be enabled when the first connecting port is not in electrical communication with the second connecting port.

12. The oral screening device having a replaceable screening head according to claim 7, wherein the first push button switch is a power switch, the second push button switch is a mode switching switch, and the third push button switch is a filming switch.

13. The oral screening device having a replaceable screening head according to claim 5, wherein the plurality of light emitting diodes 1041 is blue light emitting diodes, white light emitting diodes, violet light emitting diodes, or any combination thereof.

14. The oral screening device having a replaceable screening head according to claim 7, wherein the handle is provided with a third connecting port that is disposed on the other side of the control circuit board for connecting with an external interface.

15. The oral screening device having a replaceable screening head according to claim 14, wherein the first, second, and third connecting ports are mini USB ports, micro USB ports, or USB Type-C ports.

16. The oral screening device having a replaceable screening head according to claim 1, wherein the controller is a microcontroller unit, CPU, or digital signal processor.

17. The oral screening device having a replaceable screening head according to claim 1, wherein the handle is provided with a wireless transmitting unit that is electrically coupled with the control unit.
